(19) United States
(12) Patent Application Publication
(10) Pub. No.: US 2019/0376886 A1
(43) Pub. Date: Dec. 12, 2019

(54) Test chamber, use and method for the microbial and/or particulate barrier testing of a product

(71) Applicant: Aesculap AG, Tuttlingen (DE)

(72) Inventors: Selina Kammerer, Dauchingen (DE); Daniel Kiessling, Villingen (DE)

(21) Appl. No.: 16/425,515

(22) Filed: May 29, 2019

(30) Foreign Application Priority Data
Jun. 8, 2018 (DE) 10 2018 209 184.6

(51) Int. Cl. G01N 15/08; B01L 1/02; C12Q 1/22

(52) U.S. Cl. CPC G01N 15/0806; B01L 1/025; C12Q 1/22; G01N 2015/0846; B01L 2400/049; B01L 2300/14; B01L 2300/046; B01L 2400/0466; B01L 2200/0652

(57) ABSTRACT

A test chamber for the microbial and/or a particulate barrier testing of a number of products, the test chamber having a test space which has, at least along a straight section, an inner extent of at least 1.5 m. As a result, a particularly homogeneous microbe and/or particle distribution is achieved. The invention also relates to an associated use and to an associated method.

This application is a United States Non-provisional Application claiming priority under 35 U.S.C. § 119 from German Patent Application No. DE 10 2018 209 184.6 filed Jun. 8, 2018, the entire contents of which are herein incorporated by reference.

### DESCRIPTION

The invention relates to a test chamber for the microbial and/or particulate barrier testing of a number of products. The invention also relates to a use of a test chamber and to a method for the microbial and/or particulate barrier testing of at least one product.

A test chamber has a test space for the products, the test space being accessible by way of an opening. The test chamber also has a closing element, by means of which the opening can be closed. Furthermore, the test chamber has a microbe introducing device for introducing microbes into the test space. The test chamber has a number of pressure changing elements, which are designed to change an ambient pressure in the test space. The test space is designed to be pressure-tight within a pressure range when the opening is closed by the closing element.

Such a test chamber is known for example from the document DE 102 61 627 A1. Such test chambers may be used in particular for the microbial barrier testing of products. Such products may be in particular medical products that are provided with a germ-proof packaging. Such packagings are intended in particular to remain germ-proof even when they are exposed to the conditions of typical transporting routes that occur in practice during the transport of medical products. Such transporting operations may take place for example between a central location that performs a sterilization and various hospitals or medical practices.

Test chambers are in this case typically designed to establish microbial contamination conditions, so that it can be tested whether germs penetrate through a packaging. Serving for this purpose in particular is the microbe introducing device already mentioned at the beginning. For example, aerosols comprising microbial germs may be blown into the test space. Furthermore, typical test chambers are designed to provide different pressures, in order to simulate a realistic transporting operation, which may for example comprise changes in pressure as a result of fluctuations in air pressure or the use of lifts.

However, it has been found that uniform and reproducible microbe distribution does not occur in the test chambers known from the prior art. Rather, there are deposits of germs on walls, which considerably influences the microbe distribution. This leads to unrealistic simulation conditions, and consequently to test results that are not meaningful.

The invention addresses the problem of providing a test chamber for the microbial and/or particulate barrier testing of a number of products that has a better homogeneity of the microbe and/or particle distribution in comparison with configurations according to the prior art. Other problems addressed by the invention are those of providing an associated use and an associated method.

This is achieved according to the invention by a test chamber, a use and a method according to the respective main claims. Advantageous refinements can be taken for example from the respective subclaims. The content of the claims is made the content of the description by express reference.

The invention relates to a test chamber for the microbial and/or particulate barrier testing of a number of products. The test chamber has a test space for the products, the test space being accessible by way of an opening. The test chamber has a closing element, by means of which the opening can be closed. The test chamber has a microbe and/or particle introducing device for introducing microbes and/or particles into the test space. The test chamber has a number of pressure changing elements, which are designed to change an air pressure in the test space. The test space is designed to be pressure-tight within a pressure range when the opening is closed by the closing element.

The test chamber is distinguished according to the invention by the fact that the test space has, at least along a straight section, an inner extent of at least 1.5 m.

It has been found that the use of a correspondingly large configuration of the test space allows the achievement of a considerably better microbe and/or particle distribution, which is in particular much more uniform than is the case with small test spaces. The reason for this is in particular that the effects of deposits on walls play a considerably lesser role than in the case of test chambers known from the prior art.

The straight section may in this case be defined between any two points within the test space, so that it does not intersect a surrounding wall of the test space. Consequently, the test chamber according to the invention is considerably different from much smaller configurations according to the prior art.

The expression “number of products” is intended within the context of the present invention to be understood as meaning one product, i.e. a single product, or a plurality of products, i.e. multiple products, such as for example two, three or four products.

The expression “number of pressure changing elements” is intended within the context of the present invention to be understood as meaning one pressure changing element, i.e. a single pressure changing element, or a plurality of pressure changing elements, i.e. multiple pressure changing elements, such as for example two, three or four pressure changing elements.

The expression “product” or “products” is intended within the context of the present invention to be understood as meaning in particular a sterile product packaging or sterile product packagings and/or a packaged, in particular sterile-packaged, medical product, such as for example a surgical instrument, or packaged, in particular sterile-packaged, medical products, such as for example surgical instruments.

The expression “microbes” or “germs” is intended within the context of the present invention to be understood as meaning microorganisms, in particular pathogenic microorganisms (so-called pathogens). In particular, the expression “microbes” or “germs” is intended within the context of the present invention to be understood as meaning bacteria, such as for example Micrococcus luteus, and/or fungi and/or algae and/or viruses and/or subcellular structures, such as for example proteins and/or nucleic acids.

The particles that can be introduced into the test space may be for example dust particles, latex particles, paint particles or the like.

According to respective advantageous configurations, the test space has a width of at least 1.5 m or at least 1.8 m. According to respective preferred configurations, the test space has a height of at least 1.5 m or at least 2 m or at least 2.2 m. According to respective preferred configurations, the test space has a depth of at least 1.5 m or at least 2 m or at least 2.7 m. Such values have proven to be particularly advantageous, the advantage being all the greater the greater the respective dimension or the respective value is. Any desired combinations of the values indicated are possible.

According to respective preferred configurations, the test space has a volume of at least 3 m³, 5 m³, 8 m³, 10 m³ or 12 m³. Such values have proven to be advantageous, the advantage being all the greater the higher the value of the volume is. The microbe and/or particle distribution is all the more uniform.

Preferably, the test chamber is designed such that it can be entered on foot. This allows advantageous operability and in particular the simulation of actual keeping and/or storing conditions.

The test chamber preferably has a plurality of placement surfaces arranged one above the other, in particular in the form of at least one rack, for the products in the test space. As a result, the products can be stored on multiple levels, whereby different conditions can be realized and also a high number of products can be tested simultaneously.

The test chamber may for example also have a table or other piece of furniture with a placement surface. The advantages mentioned in the previous paragraph apply analogously.

The pressure range preferably has a lower limit which lies at atmospheric pressure (air pressure) or lies 50 mbar, 100 mbar, 150 mbar, 200 mbar, 480 mbar or 500 mbar below atmospheric pressure (air pressure). The pressure range preferably has an upper limit which lies at atmospheric pressure (air pressure) or lies 50 mbar, 100 mbar, 150 mbar, 200 mbar, 480 mbar or 500 mbar above atmospheric pressure (air pressure). It should be understood that all of the lower limits and upper limits mentioned can be combined with one another as desired. Such lower and upper limits of the pressure range have proven to be advantageous in practice.

The pressure changing elements are preferably designed to set any pressure in the pressure range when the opening is closed. As a result, the pressure can be varied over the entire range in which there is a pressure tightness.

The pressure changing elements may in particular be arranged on a rear wall of the test space, i.e. a wall lying opposite the closing element.

Preferably, a respective pressure changing element has a bellows, which is arranged in the test space and the volume of which is variable. By means of such a bellows, a pressure in the test space can be varied without it being necessary for air to be blown in or air to be blown out from the test space. As a result, an escape of microbes or a dilution of the microbe concentration in the test space can be prevented.

Preferably, the test chamber has an air supply and/or air removal system, in order to change volumes of the bellows. As a result, the bellows can be inflated or reduced in size again, so that the pressure within the test space changes correspondingly. An air supply system may for example comprise a fan or an air pump. An air removal system may for example comprise a valve, by means of which pressure can be let out, or may also comprise a fan for the active removal of air.

The test chamber preferably has a number of fans and/or a number of valves, in order to change volumes of the bellows. This has proven to be a practicable configuration.

The expression “number of fans” is intended within the context of the present invention to be understood as meaning one fan, i.e. a single fan, or a plurality of fans, i.e. multiple fans, such as for example two, three or four fans.

The closing element may in particular be configured as a pivotable door. This makes easy accessibility to the test space possible.

The microbe introducing device may be advantageously configured as an aerosol generator. This has proven to be a practicable configuration for introducing microbes. As a result, it is possible for microbes to be introduced into the test space for example by nebulizing microbes suspended in ultrapure water.

The particle introducing device may (likewise) be advantageously configured as an aerosol generator. As a result, it is possible for particles to be introduced into the test space for example by nebulizing particles suspended in ultrapure water.

The test chamber preferably also has a number of microbe collecting elements arranged in the test space. Such microbe collecting elements may in particular be designed to collect microbes present in air and/or liquids and/or on surfaces at a respective location of the test chamber or of the test space.

The expression “number of microbe collecting elements” is intended within the context of the present invention to be understood as meaning one microbe collecting element, i.e. a single microbe collecting element, or a plurality of microbe collecting elements, i.e. multiple microbe collecting elements, such as for example two, three or four microbe collecting elements.

The number of microbe collecting elements may be for example plates or dishes with a solid or liquid nutrient medium, such as for example agar, CASO agar, CASO bouillon or Columbia blood agar.

The test chamber preferably also has a number of particle collecting elements arranged in the test space. Such particle collecting elements may in particular be designed to collect particles present in air and/or liquids and/or on surfaces at a respective location of the test chamber or of the test space.

The expression “number of particle collecting elements” is intended within the context of the present invention to be understood as meaning one particle collecting element, i.e. a single particle collecting element, or a plurality of particle collecting elements, i.e. multiple particle collecting elements, such as for example two, three or four particle collecting elements.

The test chamber preferably also has a number of collecting elements arranged in the test space for collecting microbes and particles. Such collecting elements may in particular be designed to collect both microbes present in air and/or liquids and/or on surfaces and particles present in air and/or liquids and/or on surfaces at a respective location of the test chamber or of the test space.

The expression “number of collecting elements for collecting microbes and particles” is intended within the context of the present invention to be understood as meaning one collecting element for collecting microbes and particles, i.e. a single collecting element for collecting microbes and particles, or a plurality of collecting elements for collecting microbes and particles, i.e. multiple collecting elements for collecting microbes and particles, such as for example two, three or four collecting elements for collecting microbes and particles.

The test chamber preferably also has a number of microbe detecting elements, in particular on-line microbe detecting elements, arranged in the test space. Such microbe detecting elements may be designed in particular to determine a microbe concentration in air and/or liquids and/or on surfaces or a number of microbes in air and/or in liquids and/or on surfaces at a respective location of the test chamber or of the test space. As a result, microbe distributions in the test chamber or in the test space can also be determined.

The expression “number of microbe detecting elements” is intended within the context of the present invention to be understood as meaning one microbe detecting element, i.e. a single microbe detecting element, or a plurality of microbe detecting elements, i.e. multiple microbe detecting elements, such as for example two, three or four microbe detecting elements. Correspondingly, the expression “number of on-line microbe detecting elements” is intended within the context of the present invention to be understood as meaning one on-line microbe detecting element, i.e. a single on-line microbe detecting element, or a plurality of on-line microbe detecting elements, i.e. multiple on-line microbe detecting elements, such as for example two, three or four on-line microbe detecting elements.

The use of a number of on-line microbe detecting elements makes it possible with particular advantage for a determination or measurement of microbes in air and/or in liquids and/or on surfaces to take place at a respective location of the test chamber or of the test space already during a testing operation.

The number of on-line microbe detecting elements may be connected to an outer side of the test chamber, for example by way of data connections, which may be configured as solid or else wireless.

For the determination of a microbe concentration or microbe distribution, the number of microbe detecting elements may for example comprise a real-time laser-induced fluorescence system. An identification of microbes may take place here enzymatically, in particular with the aid of the enzyme NADH dehydrogenase.

The number of microbe detecting elements is preferably a number of microbe monitoring elements, in particular on-line microbe monitoring elements, i.e. a single microbe monitoring element, in particular a single on-line microbe monitoring element, or a plurality of microbe monitoring elements, in particular a plurality of on-line microbe monitoring elements, i.e. multiple microbe monitoring elements, in particular multiple on-line microbe monitoring elements, such as for example two, three or four microbe monitoring elements, in particular two, three or four on-line microbe monitoring elements. Such microbe monitoring elements are designed in particular not only to measure a microbe concentration in air and/or in liquids and/or on surfaces or a number of microbes in air and/or in liquids and/or on surfaces at a respective location of the test chamber or the test space, but additionally to monitor or check a corresponding microbe concentration or a corresponding number of microbes in the test chamber or in the test space. As a result, microbe distributions in the test chamber or in the test space can also be monitored or checked.

The use of a number of on-line microbe monitoring elements makes it possible with particular advantage for a monitoring or check of microbes in air and/or liquids and/or on surfaces to take place at a respective location of the test chamber or of the test space already during a testing operation.

Furthermore, the test chamber preferably has a number of particle detecting elements, in particular on-line particle detecting elements, arranged in the test space. Such particle detecting elements may be designed in particular to determine a particle concentration, in particular in air and/or in liquids and/or on surfaces, or a number of particles, in particular in air and/or in liquids and/or on surfaces, at a respective location of the test chamber or of the test space. As a result, particle distributions in the test chamber or in the test space can also be determined.

The expression “number of particle detecting elements” is intended within the context of the present invention to be understood as meaning one particle detecting element, i.e. a single particle detecting element, or a plurality of particle detecting elements, i.e. multiple particle detecting elements, such as for example two, three or four particle detecting elements. Correspondingly, the expression “number of on-line particle detecting elements” is intended within the context of the present invention to be understood as meaning one on-line particle detecting element, i.e. a single on-line particle detecting element, or a plurality of on-line particle detecting elements, i.e. multiple on-line particle detecting elements, such as for example two, three or four on-line particle detecting elements.

The use of a number of on-line particle detecting elements makes it possible with particular advantage for a determination or measurement of particles in air and/or liquids and/or on surfaces to take place at a respective location of the test chamber or of the test space already during a testing operation.

The number of on-line particle detecting elements may be connected to an outer side of the test chamber, for example by way of data connections, which may be configured as solid or else wireless.

The number of particle detecting elements is preferably a number of particle monitoring elements, in particular on-line particle monitoring elements, i.e. a single particle monitoring element, in particular a single on-line particle monitoring element, or a plurality of particle monitoring elements, in particular a plurality of on-line particle monitoring elements, i.e. multiple particle monitoring elements, in particular multiple on-line particle monitoring elements, such as for example two, three or four particle monitoring elements, in particular two, three or four on-line particle monitoring elements. Such particle monitoring elements are designed in particular not only to measure a particle concentration in air and/or in liquids and/or on surfaces or a number of particles in air and/or in liquids and/or on surfaces at a respective location of the test chamber or the test space, but additionally to monitor or check a corresponding particle concentration or a corresponding number of particles. As a result, particle distributions in the test chamber or in the test space can also be monitored or checked.

The use of a number of on-line particle monitoring elements makes it possible with particular advantage for a monitoring or check of particles in air and/or in liquids and/or on surfaces to take place at a respective location of the test chamber or of the test space already during a testing operation.

The number of particle detecting elements, in particular particle monitoring elements, may be for example a number of air particle counters for determining and/or monitoring a particle concentration in air or for determining a number of particles in air and/or may be a number of liquid particle counters for determining and/or monitoring a particle concentration in liquids or for determining and/or monitoring a number of particles in liquids and/or may be a number of surface particle counters for determining and/or monitoring a particle concentration on a surface or for determining a number of particles on a surface.

The expression “number of air particle counters” is intended within the context of the present invention to be understood as meaning one air particle counter, i.e. a single air particle counter, or a plurality of air particle counters, i.e. multiple air particle counters, such as for example two, three or four air particle counters.

The expression “number of liquid particle counters” is intended within the context of the present invention to be understood as meaning one liquid particle counter, i.e. a single liquid particle counter, or a plurality of liquid particle counters, i.e. multiple liquid particle counters, such as for example two, three or four liquid particle counters.

The expression “number of surface particle counters” is intended within the context of the present invention to be understood as meaning one surface particle counter, i.e. a single surface particle counter, or a plurality of surface particle counters, i.e. multiple surface particle counters, such as for example two, three or four surface particle counters.

For determining a particle concentration on a surface or for determining a number of particles on a surface, the test chamber may also have for example a number of test plates or test platelets, for example of high-grade steel.

The expression “number of test plates” or “number of test platelets” is intended within the context of the following invention to be understood as meaning one test plate or one test platelet, i.e. a single test plate or a single test platelet, or a plurality of test plates or test platelets, i.e. multiple test plates or test platelets, such as for example two, three or four test plates or test platelets.

The test plates or test platelets may be arranged on a table and/or on shelves of a rack of the test chamber. Particles adhering on the surface of the test plates or test platelets may be dissolved in an ultrasonic bath and a count of the particles in solution can subsequently be determined by means of a number of liquid particle counters.

Furthermore, the test chamber may preferably have a number of detecting elements for detecting microbes and particles, in particular a number of monitoring elements for monitoring microbes and particles, which are designed to determine, in particular to monitor, both a microbe concentration, in particular in air and/or in liquids and/or on surfaces, or a number of microbes, in particular in air and/or in liquids and/or on surfaces, and a particle concentration, in particular in air and/or in liquids and/or on surfaces, or a number of particles, in particular in air and/or in liquids and/or on surfaces, at a respective location of the test chamber or of the test space.

The detecting elements for detecting microbes and particles, in particular monitoring elements for monitoring microbes and particles, may be in particular on-line detecting elements for detecting microbes and particles, in particular on-line monitoring elements for monitoring microbes and particles.

The expression “a number of detecting elements for detecting microbes and particles, in particular monitoring elements for monitoring microbes and particles” is intended within the context of the present invention to be understood as meaning one detecting element for detecting microbes and particles, in particular one monitoring element for monitoring microbes and particles, i.e. a single detecting element for detecting microbes and particles, in particular a single monitoring element for monitoring microbes and particles, or a plurality of detecting elements for detecting microbes and particles, in particular a plurality of monitoring elements for monitoring microbes and particles, i.e. multiple detecting elements for detecting microbes and particles, in particular multiple monitoring elements for monitoring microbes and particles, such as for example two, three or four detecting elements for detecting microbes and particles, in particular two, three or four monitoring elements for monitoring microbes and particles.

Furthermore, the test chamber preferably has a suction extracting device for extracting microbes and/or particles. As a result, in particular microbes and/or particles that are still in an air of the test chamber after testing can be removed. A corresponding suction extracting device may be configured for example as a cleanroom module. As a result, it is possible with particular advantage to establish cleanroom conditions in the test space. For this purpose, it may be advantageous if the suction extracting device, in particular the cleanroom module, is configured as movable, for example in the direction of a rear wall of the test chamber.

Furthermore, the test chamber preferably has a number of fans, i.e. a single fan or multiple fans, such as for example two, three or four fans. As a result, homogeneous or else inhomogeneous microbial and/or particulate contamination conditions can be produced with particular advantage in the test space. Preferably, at least one fan is arranged on each inner side wall of the test chamber.

Fans may in particular be designed as rotorless fans. As a result, mechanical damage to or destruction of the microbes and/or particles can be prevented.

Furthermore, the test chamber may have a valve for admitting air to the test chamber.

Furthermore, the test chamber may have a filter for keeping back microbes and/or particles. As a result, contamination of the surroundings of the test chamber with the microbes and/or particles can be prevented.

Furthermore, the test chamber may have a number of UV lamps, i.e. a single UV lamp or a plurality of UV lamps, i.e. multiple UV lamps, such as for example two, three or four UV lamps, and/or a number of UV radiators, i.e. a single UV radiator or a plurality of UV radiators, i.e. multiple UV radiators, such as for example two, three or four UV radiators, for disinfecting the test space. As a result, a disinfection of the test space after the end of a testing operation is possible with particular advantage. Alternatively, the test space may be wet-disinfected after a testing operation, i.e. by means of a liquid disinfectant, which may for example be performed manually or else in an automated manner.

Furthermore, the test chamber may have a vacuum pump. As a result, in particular a removal of air before the testing operation is possible.

Furthermore, the test chamber may in particular have a cuboidal form. As a result, the simulation of actual keeping and/or storing conditions is likewise possible.

Furthermore, the test chamber may have walls of high-grade steel. Also, as a result, something analogous to storerooms, as are used for example by transport companies, hospitals or medical practices, can be produced with particular advantage.

Furthermore, a robot may be present in the test space of the test chamber. As a result, a simulation of handling activities and/or shaking movements, for example a simulation of transport, is possible. By means of the robot, a rearrangement of products for example can be achieved during testing. As a result, different positions for respective products during a testing operation can also be realized.

The invention also relates to a use of a test chamber according to the invention for the microbial and/or particulate barrier testing of a number of products. Such a test chamber in this case offers the advantages already mentioned further above also in the case of the use according to the invention. With regard to the test chamber, it is possible to fall back on all of the configurations and variants described herein.

The invention also relates to a method for the microbial barrier testing of at least one product, the method comprising the following steps:
introducing the product into a test space of a test chamber according to the invention,
closing the test space by means of the closing element,
introducing microbes and/or particles into the test space by means of the microbe and/or particle introducing device, and
changing the pressure in the test space according to a predetermined programme.

By means of the method according to the invention the microbial and/or particulate barrier testing of products can be performed in an advantageous way.

With regard to the test chamber that is used, it is possible to fall back on all of the configurations and variants described herein.

It has been realized that in the case of small test chambers, which are in particular smaller than the test chamber according to the invention, a deposit of microbes and/or particles on the walls of the test chamber takes place to a relatively great extent, and consequently the result is distorted. What is more, if the pressure is reduced, a loss of microbes and/or particles is possible.

A test sequence given by way of example is set out below:
First, a zero measurement is carried out in the pressure space during a starting phase, for example of five minutes.
Then, a microbe introducing device in the form of an aerosol generator is flushed through, for example for 10 s.
Then, the air in the test chamber is purified, for example for 5 min.
Then, a pressure change is carried out in accordance with a predetermined programme, where for example 69 bar may be set twice, 51.75 mbar may be set three times and 34 mbar may be set three times. After each pressure change, an aerosol is preferably sprayed into the test chamber.
Then, the air in the test chamber is purified, for example for 10 minutes.
Then, microbe collecting units, such as for example agar plates or vials, may be closed and the chamber may be cleaned with a disinfecting device. For this purpose, the chamber may for example also be dried for 15 min.

It is preferably provided that the air present in the pressure chamber is not changed if the pressure is increased or the pressure is reduced. Therefore, air should neither flow in from the outside nor be removed therefrom.

The pressure changing elements may in particular be designed to make possible a maximum pressure changing rate of 1.1 mbar/s. Such a value has proven to be advantageous in practice. In particular, an open-loop or closed-loop pressure control may also be provided, it being possible for example for a pressure sensor to be provided in the test chamber.

A person skilled in the art can take further features and advantages from the exemplary embodiment described below with reference to the accompanying figure and from the description then following of a sequence given by way of example of a microbial contamination testing by means of a test chamber according to the invention. In the drawing:

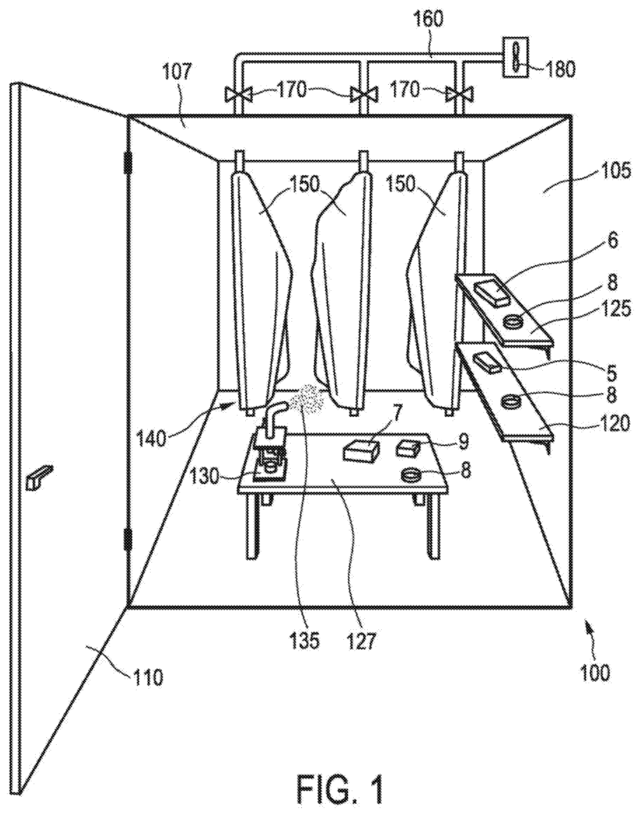

FIG. 1 shows a test chamber.

FIG. 1 shows a test chamber 100 according to an exemplary embodiment of the invention.

The test chamber 100 has a test space 105, which is accessible by way of an opening 107.

The opening 107 can be closed by means of a closing element in the form of a door 110. The door 110 is in this case pivotably secured, so that it can be opened and closed.

Arranged in the test space 105 in the present case are two shelves, which are secured on a side wall and form placement surfaces 120, 125. Stored on each of them in the present case is a product 5, 6, the products 5, 6 being intended for testing by means of a microbial and/or particulate barrier test. The products 5, 6 are in this case only schematically represented, so that it should be understood that virtually any products may be placed onto the placement surfaces 120, 125 in order to test them correspondingly.

Also, in the test space is a table 127. Stored on it is a further product 7, which is to be tested for microbial barrier properties.

When the door 110 closes the opening 107, the test space 105 is sealed in a pressure-tight and germ-proof manner. This applies at least within a predetermined pressure range, which extends from several hundreds of millibars below atmospheric pressure to several hundreds of millibars above atmospheric pressure.

Arranged in the test space 105 is an aerosol generator 130, which represents a microbe and/or particle introducing device. The aerosol generator 130 is designed to emit in the test space 105 aerosol 135, which contains microbes and/or particles.

These microbes and/or particles distribute themselves within the test space 105, so that the products 5, 6, 7 can be tested for barrier properties with respect to these microbes and/or particles. If it is found after completion of the test that corresponding microbes and/or particles have penetrated through sterile packagings of the products 5, 6, 7, the packagings clearly do not have the reliability that is required of them.

In order to simulate a change in pressure in the test space 105 during a test, there are altogether three pressure changing elements 140 located therein. Each pressure changing element 140 has a respective bellows 150. This can be externally inflated and reduced in size again. As a result, the pressure inside the test space 105 can be changed, without air having to be introduced from outside or having to be let out of the test space 105. This makes it possible to realize a change in pressure without influencing the microbe and/or particle concentration and without extracting microbes and/or particles.

For actuating the bellows 150, the test chamber 100 has a schematically represented air supply and/or air removal system 160. This is connected to the respective bellows 150 by means of a respective valve 170. The air supply and/or air removal system 160 has a fan 180, which can generate a pressure. This pressure can be selectively directed into the respective bellows 150 by way of the valves 170. As a result, a pressure in the respective bellows 150, consequently also in the test space 105, can be increased.

If the fan 180 is switched off and nevertheless a respective valve 170 is opened, the pressure in the respective bellows 150 is reduced. In this way, the pressure in the test space 105 can be both increased and reduced, so that it is possible to simulate a transporting situation for the products 5, 6, which in reality comprises for example transport by means of a truck and/or by means of an elevator, and consequently the passing through of areas with different air pressures.

For separately collecting the microbes contained in the aerosol 135, in the test space 105 there are three schematically represented petri dishes 8. These are coated with a nutrient medium, so that microbes that have entered can be collected and cultivated after carrying out the test. Each microbe in this case forms a colony, which is countable, in particular optically countable. The detection of the microbes is performed by means of a microbe detecting element 9, which is preferably designed as an on-line microbe monitoring element. As a result, it can with particular advantage already be tested during the test whether a desired number of microbes was contained in the air in the test space 105. For counting the microbe colonies, the microbe detecting element 9 may have a real-time laser-induced fluorescence system. On account of the size of the test chamber 100, it can be entered on foot.

This makes it easier for components to be taken in and out. To increase safety for operating personnel, the test chamber 100 has a detection system (not represented), which would detect a person located in the test space 105. It can in this way be ensured that a test that involves releasing microbe-containing aerosol 135 does not take place if there is a person in the test space 105.

The test space 105 has in the present case a width of 1.8 m, a height of 2.2 m and a depth of 2.7 m. Such dimensions have proven to be advantageous for typical applications. In particular, all of the dimensions mentioned are greater than 1.5 m and are consequently within a range in which a particularly homogeneous microbe and/or particle distribution has been observed. The reason for this is in particular that with such dimensions deposits on the walls are no longer as significant as in the case of small dimensions.

### EXAMPLE SECTION

### Sequence Given by Way of Example for Microbial Contamination Testing by Means of a Test Chamber According to the Invention:

First, a microbe-containing aerosol was sprayed into the test space by means of a microbe introducing device configured as an aerosol generator, so that subsequently there was a microbe suspension in all of the tubes of the aerosol generator. At the same time, the sprayed aerosol was extracted by way of a cleanroom module. After that, the test space was loaded with sterile product packagings to be tested for microbial barrier properties. For this purpose, the sterile product packagings were placed onto a table located in the test chamber.

Then it was checked by means of a detection system that there were no persons in the test chamber. After that, the door of the test chamber was closed. Next, the test was started. For this purpose, an air-admitting valve of the test space was closed. During the testing operation, a measurement by means of an on-line microbe monitoring device and a purification of the interior space of the test chamber by means of a cleanroom module took place. A microbe-containing suspension was sprayed by means of the aerosol generator over a time period of 1 minute. During the testing operation, pressure changes were carried out, for example at a maximum pressure changing rate of 1.1 mbar per second. During the pressure changes, there was no spraying of microbe-containing suspension. For carrying out the pressure changes, pressure changing elements configured as bellows were inflated by means of compressed air. A suction removal of the compressed air from the bellows was carried out by means of a vacuum pump.

After that, a microbe-containing suspension was once again sprayed over a time period of 1 minute. Then, pressure changes were once again carried out, spraying of the microbe-containing suspension not taking place during the pressure changes.

This was then followed by a rest phase for preventing air vortices. After that, a microbe-containing suspension was sprayed into the test space once again over a time period of 1 minute. Then, pressure changes were once again carried out, spraying of the microbe-containing suspension not taking place during the pressure changes.

Finally, the microbial contamination in the test chamber was reduced by way of the cleanroom module. After that, an air-admitting valve of the test chamber was opened, the microbes being kept back by means of a filter in the contamination space.

Then, the door of the test chamber was opened, and the test pieces were removed. Lastly, a disinfection (manual or automated, with a disinfecting liquid) of the test chamber was performed.

Advantages of the present invention are to be summarized (once again) as follows:

The dimensions of the test chamber according to the invention can correspond with particular advantage to actual keeping rooms and/or store rooms, it being possible for actual keeping and/or storing conditions, such as are to be encountered for example at transport companies, hospitals and medical practices, to be simulated. This also applies in particular to the relative size of products to be tested for microbial and/or particulate barrier properties and the test chamber according to the invention.

Furthermore, in the case of the test chamber according to the invention, a detection of the microbe and/or particle contamination conditions in the test space during an entire testing operation is possible with particular advantage.

Furthermore, in the case of the test chamber, an on-line detection of the microbe and/or particle contamination can be carried out during the entire testing.

Furthermore, for example a microbe- and/or particle-containing aerosol can be introduced into the test space a number of times or else the whole time during the testing. As a result, in particular a pre-defined number of microbes and/or particles can be obtained.

Furthermore, the pressure changing elements of the test chamber can be actively increased and/or reduced in size by way of an open-loop and/or closed-loop control.

A further advantage is that a plurality of products can be tested simultaneously, i.e. during one testing operation, together and in particular at the same height, for example on one placement surface of a rack, or at different heights, for example on different placement surfaces of a rack.

Furthermore, contamination of the surroundings of the test chamber with microbes and/or particles can be avoided, for example by means of a filter present in the test space.

Furthermore, the testing sequence, in particular the carrying out of pressure changes and/or the admission of a microbe- and/or particle-containing aerosol and/or a filtration of the test space air, can be automatically controlled and in particular tracked on-line.

Furthermore, constant initial conditions can be produced in the test space, for example by means of a cleanroom module located in the test space.

Furthermore, an initial microbe and/or particle contamination can be tested and detected, for example by means of an on-line microbe and/or particle monitoring device.

Furthermore, sterile test conditions can be created, in particular by means of a cleanroom module arranged in the test chamber.

Furthermore, microbes and/or particles can be kept “in suspension”, for example by producing an active and/or thermal air flow.

Furthermore, a homogeneous or directed microbe- and/or particle-contaminated flow can be produced within the test chamber by producing air flows, for example by means of fans.

Furthermore, a partial microbe and/or particle isolation can be produced by a static charging of surfaces.

Furthermore, the products to be tested for microbe and/or particle barrier properties can be rearranged during the testing operation by means of a robot arranged in the test chamber, and thus the actual handling of products, for example in hospitals or medical practices, can be simulated.

Furthermore, a disinfection (so-called “dry disinfection”) of the test chamber can be carried out for example by way of UV radiation. As a result, a rapid restoration of initial conditions suitable for carrying out a new test cycle is possible with particular advantage. Alternatively, a manual or automated disinfection with a disinfecting liquid is also possible.

1.-15. (canceled)

16. Test chamber for the microbial and/or particulate barrier testing of a number of products, the test chamber comprised of:
a test space for the products, the test space being accessible by way of an opening;
a closing element, by means of which the opening can be closed;
a microbe and/or particle introducing device for introducing microbes and/or particles into the test space;
a plurality of pressure changing elements, which are designed to change an air pressure in the test space; and
the test space being designed to be pressure-tight within a pressure range when the opening is closed by the closing element;
characterized in that
the test space has, at least along a straight section, an inner extent of at least 1.5 m.

17. The test chamber of claim 16, characterized in that
the test space has a width of at least 1.5 m or 1.8 m and/or
the test space has a height of at least 1.5 m or 2 m or 2.2 m, and/or
the test space has a depth of at least 1.5 m or 2 m or 2.7 m.

18. The test chamber of claim, 16, characterized in that the test space has a volume of at least 3 m³, 5 m³, 8 m³, 10 m³ or 12 m³.

19. The test chamber of claim 16, characterized in that the test chamber has a plurality of placement surfaces arranged one above the other for the products in the test space.

20. The test chamber of claim 16, characterized in that the pressure range has a lower limit which lies at atmospheric pressure or lies 50 mbar, 100 mbar, 150 mbar, 200 mbar, 480 mbar or 500 mbar below atmospheric pressure.

21. The test chamber of claim 16, characterized in that the pressure range has an upper limit which lies at atmospheric pressure or lies 50 mbar, 100 mbar, 150 mbar, 200 mbar, 480 mbar or 500 mbar above atmospheric pressure.

22. The test chamber of claim 16, characterized in that the pressure changing elements are designed to set any pressure in the pressure range when the opening is closed.

23. The test chamber of claim 16, characterized in that a respective pressure changing element has a bellows, which is arranged in the test space and the volume of which is variable.

24. The test chamber of claim 23, characterized in that the test chamber has an air supply and/or air removal system, in order to change volumes of the bellows.

25. The test chamber of claim, 23, characterized in that the test chamber has a plurality of fans and/or a plurality of valves, in order to change volumes of the bellows.

26. The test chamber of claim 16, characterized in that the closing element is configured as a pivotable door.

27. The test chamber of claim 16, characterized in that the microbe and/or particle introducing device is configured as an aerosol generator.

28. The test chamber of claim 16, characterized in that the test chamber also has a plurality of microbe and/or particle collecting elements and/or microbe and/or particle detecting elements, in particular on-line microbe and/or on-line particle detecting elements, arranged in the test space.

29. A test chamber of claim 16, for use in the microbial and/or particulate barrier testing of a number of products.

30. A method for the microbial and/or particulate barrier testing of at least one product, the method comprising the following steps:
introducing the product into a test space of a test chamber according to claim 16,
closing the test space by means of the closing element,
introducing microbes and/or particles into the test space by means of the microbe and/or particle introducing device, and
changing the pressure in the test space according to a predetermined program.
